(19) United States
(12) Patent Application Publication
(10) Pub. No.: US 2017/0021055 A1
(43) Pub. Date: Jan. 26, 2017

(54) MEDICAL DEVICE

(71) Applicant: THE UNIVERSITY OF MANCHESTER, Manchester (GB)

(72) Inventors: Sandra Downes, Manchester (GB); Giorgio Terenghi, Manchester (GB); Seyedeh Atefeh Mobasseri, Manchester (GB)

(21) Appl. No.: 15/107,352

(22) PCT Filed: Dec. 23, 2014
(86) PCT No.: PCT/GB2014/053835

§ 371 Date: Jun. 22, 2016

(30) Foreign Application Priority Data
Dec. 24, 2013 (GB) 1323000.8

(51) Int. Cl. A61L 27/18; C08J 5/18; B29C 65/18; B29C 39/02; B29C 39/00; B29C 39/36; A61L 27/26; A61F 2/00

(52) U.S. Cl. CPC A61L 27/18; A61L 27/26; C08J 5/18; A61F 2/0077; B29C 39/026; B29C 39/003; B29C 39/36; B29C 65/18; A61L 2430/32; C08J 2367/04; B29L 2031/753

(57) ABSTRACT

The present invention provides a peripheral nerve growth scaffold including poly-ε-caprolactone (PCL) wherein a surface of the scaffold comprises pits (blind holes) and microgrooves, the pits covering at least 5% of the surface. In embodiments, the surface has microgrooves having an average width in the range 15-20 μm, an average depth of about 5 μm, and an average spacing in the range 3-6 μm, and pits having an average area in the range 1.1-1.5 (μm)² with 20-26% coverage.

### TECHNICAL FIELD OF THE INVENTION

The present invention relates to scaffolds for peripheral nerve repair, in particular to conduits through which peripheral nerves can grow. The present invention is also concerned with methods of making such scaffolds and of their use in the repair or growth of peripheral nerves.

### BACKGROUND

The peripheral nervous system (PNS) extends outside the central nervous system (CNS) and provides the functions of, amongst other things, bringing sensory information to the CNS and receiving motor commands from the CNS, coordinating body movements and controlling the involuntary muscles. Unlike the central nervous system, the PNS is not protected by bone and is therefore vulnerable to injuries.

Damage to nerves of the PNS can cause significant motor or sensory impairment. In particular, patients with acute peripheral nerve injury usually have nerve conduction defects that can manifest as motor impairment or sensory dysfunction. Depending on the severity of the injury and the nerve affected, a severed nerve may cause paralysis, partial loss of mobility of the affected limb and/or a loss of sensation. Nerve and muscle atrophy will follow if no sufficient recovery occurs or no timely treatment is provided. Similarly, crush damage to peripheral nerves can result in reduced motor or sensory performance.

Surgical intervention is required if there is to be any prospect of repairing severed peripheral nerves. One surgical technique for attempting growth of a peripheral nerve involves providing a guide, usually in the form of a conduit, at the site of the nerve damage, to facilitate and encourage the extension of regenerating axons and prevent the scar tissue invasion. Specifically, the scaffold is selected to provide an environment that will encourage nerve growth so that nerve function can be returned. To date, success rates for peripheral nerve growth have been low and it is presently not possible to achieve the extent of peripheral nerve growth that would be required in order to repair many of the injuries experienced by peripheral nerves. It has been suggested [1] that polyhydroxybutyrate (PHB) can be used to make peripheral nerve growth conduits but, again, only low levels of peripheral nerve growth have been reported and the problem of repairing substantial peripheral nerve damage remains.

Scaffolds comprising poly-ε-caprolactone (PCL) have been proposed and show excellent biocompatibility and mechanical properties [2], [3]. Furthermore, the direction in which axons grow along and the axon organisation inside the conduits are important issues and aberrant growth of axons results in neuromas, and microgrooved surfaces within scaffolds have been shown to encourage ordered cell growth and elongations [3].

### SUMMARY OF THE INVENTION

The present inventors have noted that in order for a peripheral nerve growth scaffold to effectively facilitate growth or repair of damaged peripheral nerves, it is desirable for the scaffold to exhibit a combination of properties.

Firstly, the present inventors have found that ordered, aligned or directed growth and proliferation of neuronal and Schwann cells are important in order to optimise repair of a peripheral nerve and so the nerve conduit should provide an environment in which such behaviour can be achieved.

Secondly, the material from which the scaffold is made must be not only biocompatible but also subject to in vivo degradation at a rate which is sufficiently slow to ensure adequate time for the nerve to grow through the defect gap but fast enough to ensure that the scaffold does not remain at the site of the injury such that adequate healing can occur.

Thirdly, the present inventors have found that the mechanical properties of the scaffold must be such as to provide a robust and durable connection between the portions of the damaged peripheral nerve that is to be repaired (e.g. between proximal and distal stumps of a severed peripheral nerve), for example without breaking, swelling or collapsing once implanted. At the same time, the scaffold must exhibit sufficient flexibility to withstand handling, suturing, and surgical implantation, as well as withstand movement experienced when in situ.

Fourthly, the present inventors have found that sufficient peripheral nerve growth is only likely to occur if the scaffold is a biocompatible substrate for neuronal cells and Schwann cells. Suitably, the scaffold promotes or encourages the attachment and proliferation of peripheral nerve cells and Schwann cells. The scaffold must therefore be non-toxic and should not release harmful break-down products. The scaffold should preferably also possess surface properties that mimic the basal lamina tissue in vivo.

Fifthly, the wall thickness of the nerve conduit should be small enough to avoid neuroma formation, rigidity and tissue compression associated with a thick wall. A thin wall, along with small device size, means less allogenic biological material and faster degradation rate.

At its most general, the present invention proposes that the above criteria are addressed by providing a peripheral nerve growth scaffold that comprises poly-ε-caprolactone (PCL) and a surface which comprises microgrooves and pits adapted to encourage neuronal and Schwann cell growth. This is based on the inventors' experiments wherein enhanced ordered nerve and Schwann cell attachment and growth is observed in nerve conduits having microgrooves and pits adapted to encourage nerve and Schwann cell growth formed on a surface of a scaffold when compared to scaffolds having a smooth surface, a pitted surface, or a grooved surface that is not significantly pitted.

The present inventors have found that particular surface morphologies, that is, pits of a certain average size and/or % area coverage on the microgrooved surface, show superior effects to previously disclosed surfaces, and that by careful control of the conditions under which scaffolds as described herein are made, excellent results with respect to cell attachment, growth, and proliferation may be achieved.

Accordingly, in a first aspect, the present invention may provide a peripheral nerve growth scaffold including poly-ε-caprolactone (PCL) wherein a surface of the scaffold comprises pits (blind holes) and microgrooves, the pits covering at least 5% of the surface.

In a second aspect, the invention may provide a peripheral nerve growth scaffold including poly-ε-caprolactone (PCL) wherein a surface of the scaffold comprises pits (blind holes) and microgrooves, the pits having an average area equal to or greater than 0.5 (μm)². Preferably, these pits cover at least 5% of the surface.

As used herein, and unless stated otherwise, the surface refers to the surface of the scaffold having the microgrooved pattern.

The present inventors have found that the combination of microgrooves and pits tailored to the microgrooves on a surface of a scaffold comprising PCL surprisingly exhibits not only excellent mechanical properties and biocompatibility with peripheral in nerve cells and Schwann cells but also provides a more favourable environment for nerve and Schwann cell growth and proliferation than microgrooves alone. The present inventors attribute these enhanced properties to a co-operative or even synergistic effect arising from the combination of pits and microgrooves on the surface.

Previous teaching has indicated that scaffolds and conduits having smooth grooved surfaces result in ordered nerve and Schwann cell growth [3]. The microgrooves were provided on an inner (luminal) surface of the conduit, with a smooth (non-grooved) outer surface taught as desirable. Surprisingly, the present inventors have found that a significant level of pitting on this inner surface actually enhances cell proliferation, particularly in the initial days of cell growth. The present inventors have further found that certain optimal levels of pitting and/or pits of certain average sizes can aid cell attachment and/or facilitate nerve and Schwann cell growth and proliferation within the grooves. This increased roughness of the grooves does not, as may be expected, negatively impact on cell growth and proliferation within the grooves. Indeed, as demonstrated herein, the provision of comparatively substantial amounts of pitting and/or pits of substantial size has a positive effect on cell growth performance, despite the obvious “degradation” of the microgroove structure.

Furthermore, the present inventors have found that for grooved and pitted surface scaffolds according to the present invention provided as conduits, significant levels of pitting on an outer surface, both in terms of average pit size and % pitted area coverage, does not adversely affect in vivo performance in terms of tissue adhesion or inflammation. Again, this is contrary to previous teaching which, as noted above, sought to preclude such pitting in favour of a smooth surface.

The present inventors have found that the nature of the pits can be controlled through careful selection of the conditions under which the scaffolds are made. Certain combinations of microgrooves and pits are preferred and these combinations exhibit further enhanced properties in terms of cell attachment, alignment, and/or proliferation, and balanced mechanical properties. Disclosed herein are certain preferred combinations found by the present inventors to demonstrate superior effects.

Preferably, at least 10% of the microgrooved surface is pitted, more preferably at least 15%, most preferably at least 20%. The present inventors have found that increased pitting improves cell proliferation. Very high % pitted area, especially in combination with large pit sizes, may result in difficulties in cell attachment and decreased mechanical strength. Accordingly, preferably, the surface is not more than 80% pitted, more preferably, not more than 60% pitted, more preferably not more than 40% pitted, most preferably not more than 30% pitted. The method of measuring % pitted area is described herein.

In some embodiments, the % coverage of the pits on the microgrooved surface is in the range 18-30%. Preferably the % coverage of the pits on the microgrooved surface is in the range 20-26%, more preferably, 21-25%, most preferably 22-24%. In some preferred combinations, the % coverage of the pits on the microgrooved surface is about 23%. % Pitted area coverage of these levels has been shown to demonstrate excellent cell attachment, growth, and proliferation. This balance of challenging performance criteria is impressive, particularly as good mechanical properties are also achieved.

Appropriate selection of suitable average pit size may also improve the observed co-operation/synergy. Preferably, the pits on the microgrooved surface have an average area equal to or greater than 0.5 (μm)², preferably equal to or greater than 0.7 (μm)², more preferably equal to or greater than 0.9 (μm)². The present inventors have found that pits of at least these sizes enhance cell proliferation. Very large pit sizes, especially in combination with higher % pitted area coverage, may result in poorer initial cell attachment while still demonstrating good cell proliferation. Accordingly, preferably the average pit size is equal to or less than 4.0 (μm)², more preferably, equal to or less than 3.0 (μm)², more preferably, equal to or less than 2.0 (μm)², more preferably equal to or less than 1.5 (μm)². The method of measuring average pit size is described herein.

In some preferred combinations, the pits on the grooved surface have an average area in the range 0.9-3.0 (μm)², preferably in the range 1.0-1.5 (μm)², more preferably 1.1-1.5 (μm)², more preferably 1.1-1.3 (μm)², most preferably about 1.2 (μm)². The presence of pits of these average sizes has been shown to demonstrate excellent cell attachment, growth, and proliferation.

The microgrooves form or are part of a pattern, for example an array of microgrooves. Suitably the pattern or array comprises a plurality of microgrooves arranged side-by-side and are aligned along the lengthwise or “long” direction of the scaffold. Thus, suitably, when the scaffold is inserted in vivo so as to bridge a gap in a peripheral nerve, the microgrooves are aligned between the respective ends of the damaged nerve. In other words, it is preferred that the microgrooves are aligned in the direction of intended nerve growth. In the case of a tubular conduit scaffold, the microgrooves are preferably aligned substantially parallel to the longitudinal axis of the conduit. These may be described as longitudinal microgrooves.

Preferably the microgrooves extend to one or both ends of the scaffold, preferably both ends (the scaffold ends in use being associated with respective ends of the damaged nerve). Preferably, the microgrooves extend along at least 50% of the length of the scaffold, more preferably at least 60%, more preferably at least 70%, more preferably at least 80%, more preferably at least 90% and most preferably substantially all of the length of the scaffold.

Preferably the microgrooves are provided on at least 50% of the inner surface, by area, of the scaffold (e.g. conduit). That is, suitably the pattern comprising the microgrooves (i.e. the microgrooves themselves and the ridges or spaces between the microgrooves) is provided on at least 50% of the inner surface of the scaffold. More preferably the microgrooves are provided on at least 60% of the inner surface of the scaffold, more preferably at least 70%, more preferably at least 80%, more preferably at least 90%, more preferably at least 95% and more preferably substantially all of the inner surface.

The width of the microgrooves and the spacing of the microgrooves (i.e. the ridge between neighbouring microgrooves) can be selected independently. The width and the spacing can be the same or different. Preferably the width and the spacing are different. Suitably the width is larger than the spacing. Preferably the width is at least 1.5 times the spacing, more preferably at least 1.75 times the spacing, more preferably at least 2 times the spacing, more preferably at least 2.5 times the spacing.

Whilst there is no particular upper limit, a maximum ratio of 10:1, preferably 8:1, preferably about 6:1 is preferred.

A particularly preferred range is about (width:spacing) 2:1 to 8:1, more preferably about 2.5:1 to 6.5:1, more preferably about 3:1 to 5:1.

Preferably the microgrooves are substantially continuous, i.e. unbroken. Nevertheless, in embodiments some breaks or discontinuity may occur without a substantial adverse effect on the performance of the scaffold.

Suitably the width of the microgrooves is at least 2 μm, preferably at least 4 μm, more preferably at least about 5 μm, more preferably at least 8 μm, more preferably at least about 10 μm, more preferably at least 13 μm, more preferably at least 15 μm. In embodiments, a groove width of about 15 μm was found to provide excellent results. But larger widths are possible, for example up to 50 μm.

Preferably the width of the microgrooves is 50 μm or less, preferably 40 μm or less, more preferably 30 μm or less and most preferably 25 μm or less.

Preferably, the microgrooves have an average width in the range 2 μm to 40 μm, more preferably 4 μm to 35 μm, more preferably 8 μm to 25 μm and even more preferably about 10 μm to about 20 μm.

A particularly preferred width of the microgrooves is 10-40 μm, more preferably 15-35 μm, more preferably, 15-25 μm, most preferably, 15-20 μm.

Suitably the width of each microgroove is substantially the same. Thus, suitably each microgroove in the pattern or array has substantially the same width.

Preferably the spacing between the microgrooves is 30 μm or less, more preferably 25 μm or less, more preferably 20 μm or less, more preferably 15 μm or less, more preferably 10 μm or less and most preferably 8 μm or less. Suitably, the spacing between the microgrooves is at least 2 μm, preferably at least 3 μm. A particularly preferred spacing is 3-6 μm.

Suitably the spacing between each pair of microgrooves is substantially the same. Thus, suitably each spacing (e.g. ridge between microgrooves) in the pattern or array is substantially the same.

The depth of the microgrooves can be selected independently of the width and/or spacing of the microgrooves. However, in embodiments, as discussed above, the depth can be related to the spacing and/or width. For example, where etching is used to create the grooves (e.g. by etching a template), groove width may increase as a function of increasing depth.

Suitably the depth (as measured at the deepest point in the case of a microgroove that has a contoured or profiled cross-section) is at least 1 μm, preferably at least 2 μm, more preferably at least 3 μm, more preferably at least 4 μm and most preferably at least about 5 μm.

A suitable upper limit for the depth is 10 μm, preferably 8 μm.

A particularly preferred range for the depth is 4 to 6 urn, with about 5 μm being especially preferred.

Suitably, the depth of each microgroove is substantially the same. Thus, suitably each microgroove in the pattern or array has substantially the same depth. Preferably, the microgrooves have an average width in the range 15-20 μm, the width being measured at the top (widest point) of the microgrooves. Preferably, the microgrooves have an average depth in the range 3-7 μm, more preferably an average depth of about 5 μm. Preferably, the microgrooves have an average spacing in the range 3-6 μm. In some preferred combinations, the microgrooves have an average width in the range 15-20 μm, an average depth of about 5 μm, and an average spacing in the range 3-6 μm.

The cross-section (taken perpendicular to the longitudinal axis of the grooves) or profile of the microgrooves can have any desired shape, for example rectangular (i.e. vertical sides and a flat bottom). However, the present inventors have found that a microgroove having sloping or non-vertical sides can give rise to particularly good cell growth performance, especially cell alignment. In particular, a V-shaped, especially a truncated V-shaped, cross-section is preferred.

Accordingly, preferably, the microgrooves have a cross-section having a sloping wall, for example a sloping wall having an angle of at least 20° to the vertical, preferably at least 30°, more preferably at least 40°, more preferably at least 50° and most preferably about 55°. Preferably both side walls are at an angle selected from the above. Suitably both side walls have the same angle. The cross-section, as referred to herein, describes the cross-section taken perpendicular to the longitudinal direction of the grooves. Preferred cross-sections include V-shaped and truncated V-shaped. In the case of a tubular conduit scaffold, the slope of the wall with respect to the vertical is defined with respect to the radial direction. In other words, the vertical is defined with respect to the surface when laid out flat (e.g. in practice, prior to forming a conduit).

Suitably, the scaffold includes at least 50 wt % PCL, based on the total weight of the scaffold. Preferably the scaffold includes at least 60 wt % PCL, more preferably at least 70 wt %, more preferably at least 75 wt % and most preferably about 80 wt % PCL.

The PCL as used herein can be PCL homopolymer or PCL copolymer.

If the PCL is present as a PCL copolymer, it is preferred that the PCL monomer is present in an amount of at least 50 wt % of the copolymer, based on the total weight of the polymer. Preferably at least 60 wt % of the copolymer is PCL monomer, more preferably at least 70 wt % most preferably at least 80 wt %.

The present inventors have found that the degradation rate and/or the peripheral nerve cell adhesion properties of the scaffold can be further improved if the scaffold also includes polylactic acid (PLA). Suitably, the nerve growth scaffold also includes polylactic acid (PLA).

Suitably, if the PLA is provided as a copolymer, i.e. as PCL-PLA copolymer, PCL and PLA are the only comonomers. However, further comonomers can also be present. The PLA may be provided as a co-polymer with PCL, or the two polymers may be provided as a blend. Preferably, the PCL and PLA are provided as a blend.

Preferably no more than 50 wt % of the scaffold is PLA, more preferably no more than 40 wt %, more preferably no more than 30 wt % and more preferably no more than 25 wt %. A particularly preferred concentration of PLA is about 20 wt %, that is, the PCL and PLA are provided in a w/w ratio of about 8:1 to 2:1, preferably about 6:1 to about 3:1, most preferably about 4:1. This has been found to provide a good balance of mechanical and cell adhesion properties.

The term “PCLA” is used herein to denote a combination of PCL and PLA. PCLA can be a mixture (blend) of PCL and PLA, or a PCL-PLA copolymer.

Suitably the PCL has a number average molecular weight (Mn) in the range 10,000 to 200,000. Preferably the Mn is in the range 20,000 to 140,000, more preferably 40,000 to 120,000 and most preferably 60,000 to 100,000. A particularly preferred Mn is about 80,000.

Suitably the PLA, if present, has a number average molecular weight (Mn) in the range 10,000 to 100,000. Preferably the Mn is in the range 10,000 to 80,000, more preferably 10,000 to 50,000 and most preferably 20,000 to 40,000. A particularly preferred Mn is about 30,000.

Scaffolds as described herein are formed as films on a patterned template, the template having a pattern that is the negative of the desired microgroove pattern. The films are cast onto the substrate as a solution of PCL, and PLA if present, in solvent, with the solvent evaporated under controlled conditions. It is this controlled evaporation that leads to the formation of suitable pits on the grooved surface. In certain preferred combinations, the substrate has microgrooves having an average width in the range 15-20 μm, an average depth in the range 3-7 μm, and an average spacing in the range 3-6 μm, and pits having an average in the range 0.9-3.0 (μm)² with 18-30% coverage. More preferably, the surface has microgrooves having an average width in the range 15-20 μm, an average depth in the range 3-7 μm, and an average spacing in the range 3-6 μm, and pits having an average area in the range 1.1-1.5 (μm)₂ with 20-26% coverage. In some embodiments, the surface has microgrooves having an average width in the range 15-20 μm, have an average depth in the range 3-7 μm, and an average spacing in the range 3-6 μm, and pits having an average area of about 1.2 (μm)² with about 23% coverage. A particularly preferred microgroove depth is about 5 μm.

Suitably the edges of the film may be fixed together. Preferably this is achieved by heat sealing the film in its rolled up state. For example, the rolled up film (suitably on the conduit forming member) is heat sealed. Preferably heat sealing is achieved using a hot plate, but other heat sources could be used. Thus, a conduit is suitably formed by rolling up a film and heat sealing the edges of the film. Suitably heat sealing occurs at a temperature in the range 30-50° C., for example about 40° C. In practice, the heat sealing temperature is selected based on the melting temperature (Tm) of the material. Melting temperature can be measured by DSC, for example. Other fixing methods can also be used. However, heat sealing is preferred, not least because the surface morphology of the film, including the microgrooves is maintained after heat treatment. A further advantage of this approach is that no other potentially toxic materials (e.g. super glue) are introduced to this system by using the heat sealing method.

Scaffolds as described herein may be formed by casting films in the form of a solution onto a patterned template, then controlling the rate of solvent evaporation to result in the formation of pits of the desired size and pitted area coverage. Suitably, the solvent is a halogenated solvent, for example, a halogenated C₁₋₁₀ alkane or alkane. Chlorinated C₁₋₄ alkanes, especially chloro-substituted methane, are especially preferred. The most preferred solvents are dichloromethane (DCM) and chloroform. DCM is particularly preferred as the present inventors have found that the evaporation of DCM can be easily controlled as described herein to produce a microgrooved pitted surface having desirable properties.

Suitably, the thickness of the film is in the range 10 μm to 300 μm. Preferably, the thickness of the film is in the range 10 μm to 200 μm, more preferably 10 μm to 100 μm, more preferably, 20 μm to 100 μm, more preferably, 20 μm to 80 μm, more preferably 50 μm to 80 μm, and most preferably 60 μm to 70 μm.

The microgrooved and pitted surface of the resultant film is formed at the surface side (SS) of the film. Pits can also be formed at the air side (AS) of the film. These pits are typically larger, and the AS may have a greater % pitted area coverage, than the pits on the microgrooved (SS) surface. Suitably, when the scaffold is provided as a conduit, the microgrooved and pitted surface side (SS) of the cast film forms an inner (luminal) surface, while the more pitted air side (AS) forms the outer surface of the conduit.

As described herein, the pit size and % pitted area coverage may be influenced by a variety of factors apparent to the skilled person. These may include the conditions under which the solvent evaporates, the thickness of the film as cast and the properties of the solution used to cast the film (the concentration and composition of the biocompatible polymers, the viscosity, the solvent(s) used).

Through careful selection of the humidity conditions in which the solvent evaporates, a microgrooved surface having pits of the desired size and/or desired % pitted area coverage may be obtained. Similarly, through selection of an appropriate temperature under which the solvent evaporates, the size and/or % pitted area coverage may be controlled. Slower evaporation under, for example, higher relative humidity conditions, results in larger pit sizes and great % pit coverage on both the AS and SS of the cast film. Temperature and humidity may be controlled by casting the films in an environmental chamber set to maintain suitable conditions. Methods and equipment for controlling atmospheric conditions, for example within such a chamber, are known in the art.

Suitably, the temperature and humidity conditions are selected to complement the microgrooved pattern, the thickness and the properties of the solution, in order to arrive at a microgrooved surface having pits of the desired size and/or % pitted area coverage.

The solution may be in a chloro-methane solvent, preferably in DCM. Suitable concentrations of biocompatible polymer (PCL, PLA and/or other if present) in the solvent may be in the range 1 to 10% w/v, preferably 1 to 5% w/v, more preferably 2 to 4% w/v. A particularly preferred concentration is about 3% w/v.

Suitably, the scaffold is in the form of tubular conduit, with the microgrooved and pitted surface forming the inner (luminal) surface of the conduit. Preferably, the tubular conduit has a single tubular conduit wall defining an inner (luminal) space (i.e. the lumen, cavity or channel within the conduit).

Suitably the conduit has a circular cross-section.

Preferably the conduit is substantially straight. However, the conduit can also be bent or curved.

Suitably, the tubular conduit wall of the tubular conduit is of substantially uniform thickness about the circumference of the tubular conduit and/or along the length of the tubular conduit. Suitably, the thickness of the tubular conduit wall is in the range 10 μm to 300 μm. Preferably, the thickness of the tubular conduit wall is in the range 10 μm to 200 μm, more preferably 10 μm to 100 μm, more preferably, 20 μm to 100 μm, more preferably, 20 μm to 80 μm, more preferably 50 μm to 80 μm, and most preferably 60 μm to 70 μm.

When the scaffold is a conduit, it is preferred that the tubular conduit wall is not porous, that is, it has only very few, or more preferably no, pores extending through the thickness of the wall (through holes). This arrangement has been found to provide advantages because it prevents the escape of regenerating axons from the conduit. It may also help to prevent ingrowth of fibrous tissues which can lead to unwanted scarring. This may assist in providing a controlled environment within the conduit for nerve repair. However, a small number of such pores may be present. If present, these pores may assist in avoiding the building up of pressure resulting from fluid retention.

Preferably no more than 2%, more preferably no more than 1%, of the surface area comprises such pores. Suitably, if such pores are present, they have a diameter of not larger than 15 μm, preferably in the range 1-10 μm.

The scaffold may be used to treat peripheral nerve damage.

Suitably the length of the scaffold, e.g. the conduit, is selected to be appropriate to the nerve damage that is to be repaired. For example, if the peripheral nerve damage comprises a severed peripheral nerve with 10 mm of the peripheral nerve missing, then the length of the scaffold will be chosen so as to be sufficient to bridge the gap in the peripheral nerve. Typically, the conduit will be longer (e.g. 10% to 50% longer) than the gap.

Typically, the scaffold has a length in the range 5 mm to 50 mm, more preferably 5 mm to 30 mm, most preferably 5 mm to 20 mm. As discussed above, preferably the microgrooves extend along at least 50%, preferably substantially all of this length.

As with the length of the scaffold, the width (measured from the outer surface of the scaffold), e.g. the diameter, of the scaffold is selected so as to be appropriate to the peripheral nerve damage that is to be repaired. Suitable diameters are in the range about 1 to about 5 mm. For example, the scaffold may be a tubular conduit having an inner (luminal) space surrounded and defined by a tubular conduit wall, the inner luminal space having a diameter in the range about 1 to about 5 mm. Suitably, the conduit has only a single lumen (channel).

Peripheral nerve damage can be a gap in a peripheral nerve, i.e. a severed peripheral nerve. Alternatively or additionally, peripheral nerve damage can be a partially severed peripheral nerve. Alternatively or additionally, peripheral nerve damage can be a crushed peripheral nerve.

Suitably the scaffold provides a microenvironment at the injured site with protecting and promoting effects for the regenerating peripheral nerve. For example, it can prevent the infiltration of fibroblasts and the escape of regenerating neurites; at the same time it can contain endogenous growth factors in situ. Therefore, the scaffold is suitable for treating crushed/damaged peripheral nerves as well as severed peripheral nerves.

In particular, the scaffold of the present invention can be used to treat neurapraxia (nerve nonfunction), axonotmesis (axon cutting), and neurotmesis (nerve cutting).

It is envisaged that the scaffold of the present invention is used to treat some or all of these types of peripheral nerve damage.

The scaffold is preferably used to treat acute peripheral nerve injury.

The peripheral nerve damage can occur as a result of accidental injury, disease or surgical procedures. For example, peripheral nerve damage can occur as a result of a cut to the hands or feet, crush injuries, organ transplant, tumour removal, congenital birth defects or previous attempts to repair peripheral nerves.

The scaffold of the present invention can be used to repair peripheral nerve damage wherever it occurs in the body. Examples of peripheral nerves that are most frequently damaged include: palmar digital nerves, median nerves, the ulnar nerve and the radial nerve. Further examples include the brachial plexus and musculocutaneous nerves. Yet further examples (in the lower limbs) include plantar digital nerve, peroneal and the sciatic nerve. The advantageous mechanical properties observed by the present inventors for scaffolds according to the present invention result in scaffolds that are suitable for treating peripheral nerve damage in areas of relatively high flexibility and movements, for example, in the limbs and extremities.

In embodiments, the scaffold is used to enclose the affected part of the peripheral nerve (i.e. the damaged portion).

In other embodiments wherein the peripheral nerve damage includes a severed peripheral nerve such that there is a gap in the peripheral nerve, the scaffold is positioned so as to bridge the gap between the respective proximal and distal ends of the severed nerve. In preferred embodiments wherein the scaffold is a conduit, the conduit is positioned so as to provide a guide for peripheral nerve growth between the axial and distal ends of the severed nerve.

The scaffold can be attached to the peripheral nerve by any means known to the skilled reader. Suitably the scaffold is attached using a suture. Suitably the suture provides attachment between the epineurium and the scaffold. Bioglue can also be used.

The scaffold can be used to treat peripheral nerve damage in an animal, including humans and non-humans. Treatment of humans is particularly preferred.

To assist in the treatment of peripheral nerve damage, the scaffold may be used in conjunction with a peripheral nerve cell growth medium (e.g. a gel matrix). For example, the peripheral nerve cell growth medium includes growth factors and Schwann cells. Suitably the peripheral nerve cell growth medium is a transport media for cells and/or growth factors (i.e. the peripheral nerve cell growth medium is, or comprises, a transport matrix). Typically the peripheral nerve cell growth medium is a hydrogel.

In use, the peripheral nerve cell growth medium (e.g. a gel matrix) is preferably introduced into the scaffold in situ. Typically the scaffold is positioned at the site of the peripheral nerve damage (e.g. after suturing) and then the growth medium (gel matrix) may be delivered to the scaffold. In preferred embodiments wherein the scaffold is a conduit, suitably the growth medium is delivered into the luminal volume of the conduit, optionally together with cells and/or growth factors. Suitably this is achieved by injecting the growth medium, for example through the end of the conduit after suturing. Alternatively, the Schwann cells or stem cells could be introduced to the scaffold prior of implantation.

While the invention has been discussed above in relation to a scaffold, the present invention also provides methods and uses relating to the scaffold.

At its most general, a further aspect of the present invention relates to method of making a peripheral nerve growth scaffold formed a cast film formed by casting a suitable solution including poly-ε-caprolactone (PCL) onto a patterned template, the template having a pattern that is a negative of the microgroove pattern, and then evaporating the solvent under controlled temperature and humidity conditions, suitably in an environmental chamber, to control the formation of pits at the surface.

Accordingly, in a further aspect, the present invention may provide a method of making a peripheral nerve growth scaffold as described herein, the method comprising the step of:
i) selecting a template forming a negative of a microgroove pattern;
ii) applying a solution comprising poly-ε-caprolactone (PCL) to the template;
iii) evaporating the solvent to form a film under controlled temperature and humidity conditions, suitably in an environmental chamber, to control the formation of pits at the surface; and
iv) removing the film from the template.

Preferably the concentration of the PCL in the solvent is in the range 1 to 10% (wt/vol), more preferably 1 to 5%, and most preferably 2 to 4%. A particularly preferred concentration is about 3%.

Chlorinated solvents are preferred. Chloro-substituted C₁₋₄ alkanes, especially chloro-substituted methane, are especially preferred. The most preferred solvent is dichloromethane (DCM). Suitably the solvent is heated, for example to a temperature in the range 40-60° C. This may assist in dissolving the PCL.

The solution may also include polylactic acid (PLA). Preferably the weight ratio of PCL:PLA in the solution is in the range 20:1 to 1:1. More preferably the ratio is in the range 10:1 to 2:1, more preferably 7:1 to 2:1, more preferably 6:1 to 2:1 and most preferably 5:1 to 3:1. A particularly preferred ratio is about 4:1.

The template may be a silicon and/or silica (silicon dioxide) template, for example, a patterned silicon wafer. Suitably the template is cleaned to remove impurities (e.g. degreased) prior to casting.

Preferred microgroove patterns are described herein. An especially preferred microgroove pattern has microgrooves having an average width in the range 15-20 μm, an average depth of in the range 3 to 7 μm, and an average spacing in the range 3-6 μm.

Preferably the PCL solution is applied directly to the template, i.e. without an intervening layer.

Factors that may determine suitable conditions for producing pits of desirable size and/or % pitted area coverage are described herein.

Suitably the conditions are selected to result in a % pitted area coverage of at least 5% on the microgrooved surface. Preferably, the conditions are selected such that at least 10% of the microgrooved surface is pitted, more preferably at least 15%, most preferably at least 20%. As very high levels of pitted area coverage, especially in combination with large pit sizes, may result in difficulties in cell attachment, preferably the conditions are selected such that the surface is not more than 80% pitted, more preferably, not more than 60% pitted, most preferably not more than 40% pitted.

Under preferred conditions, the % coverage of the pits on the microgrooved surface is in the range 18-30%. Preferably the % coverage of the pits on the microgrooved surface is in the range 20-26%, more preferably 21-25%, most preferably 22-24%. In some preferred combinations, the % coverage of the pits on the microgrooved surface is about 23%.

Suitably the conditions are selected to, in addition to resulting in the desired % pitted area coverage, result in pits of a desired average size. The inventors have found that appropriate selection of suitable average pit size improves cell attachment, growth, and proliferation. Suitably, the conditions are selected to result in pits on the microgrooved surface having an average area equal to or greater than 0.5 (μm)², preferably equal to or greater than 0.7 (μm)², more preferably equal to or greater than 0.9 (μm)².

The present inventors have found that very large average pit size reduces cell attachment on the microgrooved surface. Accordingly, the conditions may be selected to afford an average pit size that is equal to or less than 4.0 (μm)², more preferably, equal to or less than 3.0 (μm)², more preferably, equal to or less than 2.0 (μm)², more preferably equal to or less than 1.5 (μm)².

Under some preferred conditions, the pits on the grooved surface have an average area in the range 0.9-3.0 (μm)², preferably in the range 1.0-1.5 (μm)², more preferably, 1.1-1.5 (μm)², most preferably in the range 1.1-1.3 (μm)². An especially preferred size is about 1.2 (μm)².

In embodiments of the solvent casting method described herein, biocompatible polymers can be simply dissolved and poured onto a master (template), cured under controlled conditions to facilitate suitable pit formation, and removed. Many replicates (copies) can be fabricated from a single patterned master; as such the cost and processing time can be minimized

Preferably, the film is washed after solvent evaporation is completed. Suitable washing agents include water, preferably distilled water.

Preferably the film is sterilised, for example sterilised using UV radiation, γ radiation or 70% ethanol. Indeed, any suitable known technique for sterilising can be used. UV radiation is preferred.

The method may further comprise the step of joining opposite edges of the film together by heat sealing as described herein to form a tubular conduit having the surface of the film having pits and microgrooves as an inner (luminal) surface.

Preferably the scaffold is flexible. In embodiments, the present inventors have found that the PCL scaffolds described herein are highly flexible. This flexibility reduces or avoids irritation to surrounding tissues. This flexibility also facilitates efficient use of the scaffolds in locations of comparatively high motility, for example, in limbs and extremities.

Preferably the film used to form the scaffold has a tensile strength of at least 1 MPa. Preferably the film used to form the scaffold has a maximum strain of at least 1 mm/mm.

In a further aspect, the present invention provides a kit for treating a peripheral nerve in a human or animal, the kit including a peripheral nerve growth scaffold according to any one of the preceding claims.

Preferably, the kit includes the peripheral nerve growth scaffold in a sterilised package.

Preferably, the kit includes a plurality of peripheral nerve growth scaffolds as described herein. More preferably, the peripheral nerve growth scaffolds vary in size according to one or more of the following dimensions: scaffold length and scaffold internal diameter. A user may then select the correct size of nerve repair scaffold from the kit to suit the requirements of a particular nerve repair treatment. Suitably, each peripheral nerve growth scaffold in the kit is in an individual sterilised package.

In a further aspect, the present invention provides a method of treating a damaged peripheral nerve using a peripheral nerve growth scaffold as described herein.

In a further aspect, the present invention provides a method of treating a severed peripheral nerve, the method including the steps of
(i) providing a peripheral nerve growth scaffold as described herein,
(ii) coupling a first severed end of the nerve to a first portion of the scaffold, and
(iii) coupling a second severed end of the nerve to a second portion of the scaffold,
wherein the first and second portions of the scaffold are separated by a growth portion of the scaffold having a growth surface on which at least one of the first and second severed ends of the nerve is able to grow in a direction towards the respective other severed end.

Suitably the first severed end is the proximal end of the nerve and the second severed end is the distal end of the severed nerve. Conduits as described herein may be used to encase an area in which cell regeneration is desired, that is, by providing a conduit in which nerves cells may grow, thereby reconnecting first and seconds ends of the severed nerve. As described herein, suitably the conduit is longer than the gap to be bridged. Therefore, suitably the conduit may encase the severed ends, thereby forming a closed conduit between them.

Any one or more of the aspects of the present invention may be combined with any one or more of the other aspects of the present invention. Similarly, any one or more of the features and optional features of any of the aspects may be applied to any one of the other aspects. Thus, the discussion herein of optional and preferred features may apply to some or all of the aspects. In particular, optional and preferred features relating to the scaffold, methods of making the scaffold and methods of using the scaffold, etc apply to all of the other aspects. Furthermore, optional and preferred features associated with a method or use may also apply to a product (e.g. scaffold) and vice versa.

### BRIEF DESCRIPTION OF THE DRAWINGS

Embodiments of the invention and experiments illustrating the advantages and/or implementation of the invention are described below, by way of example only, with respect to the accompanying drawings, in which:

FIG. 1 shows SEM images of the grooved surface cast PCL/PLA films on a grooved silicon template (width 15-20 μm; depth 5 μm; space 3-6 μm) under different environmental conditions: (a) Type I; (b) Type II; (c) Type III.

FIG. 2 shows SEM images of the air surface of cast PCL/PLA films on a grooved silicon template (width 15-20 μm; depth 5 μm; space 3-6 μm) under different environmental conditions: (a) Type I; (b) Type II; (c) Type III.

FIG. 3 shows the SEM imaging processing steps used in the measurement of pit size and pitted area coverage; (a) SEM image converted to 8-bit; (b) after application of threshold; (c) automatic measurement by software.

FIG. 4 shows SEM images of casting PCL/PLA films on silicon (width 20-25 μm; depth 10 μm; space 20-25 μm) under different environmental conditions: (a) Type I; (b) Type II; (c) Type III.

FIG. 5 shows the maximum stress values measured for the Type I, Type II, and Type III films.

FIG. 6 shows the stress values measured for the Type I, Type II, and Type III films.

FIG. 7 shows SEM images showing cell fixation on PCL/PLA films. a) Type I; (b) Type II; (c) Type III on day 5 from an initial cell seeding density 3000 cell/ml.

FIG. 8 shows fluorescence micrograph images of NG108-15 on PCL/PLA films on day 5, initial cell seeding density 3000 cell/ml. F-actin was stained in green (phalloidin) and nucleus were stained in blue (DAPI) (a) Type I; (b) Type II; (c) Type III (×20); (d) control (×20), (Cells growing on the smooth glass slide).

FIG. 9 shows cellular proliferation on Type I, Type II, and Type III PCL/PLA films over the course of 5 days.

Statistical analysis was carried out by GraphPad Prism 4 (GraphPad Software Inc CA, USA). A one-way ANOVA was used to determine the statistical difference between data, *p<0.05, **p<0.01 and ***p<0.001.

### DETAILED DESCRIPTION OF THE INVENTION

### Definitions

The term “scaffold” as used herein is well known to the skilled reader. In particular, a scaffold in the context of the present invention is a structure adapted for peripheral nerve growth. Suitably the scaffold promotes or enhances peripheral nerve growth.

The term “pit” as used herein means a closed-end pore or “blind” hole. In short, a “pit” as used herein does not extend all of the way through the thickness of the scaffold. For example, when the scaffold is a tubular conduit, a “pit” as used herein does not extend all the way through the thickness of the tubular conduit wall. Suitably, the pit may extend not more than 50% through the thickness of the conduit wall, for example, not more than 40%, 30%, 20%, 10%, or 5% through the thickness of the conduit wall.

The term “microgroove” as used herein means a groove having a width of at least 1 μm. In this connection, the skilled reader will understand “groove” to include elongate channels and trenches formed on or in the surface of the scaffold material.

### Film Formation

PCL pellets (Sigma-Aldrich Mn ˜70,000-90,000 g/mol) and PLA (Mn ˜30,000 g/mol, Mw ˜60,000) were dissolved in a ratio of 4:1 in dichloromethane (3.0%, wt/v). PCL/PLA solution was evenly applied onto a silicon substrate (25 mm×25 mm) 1.0 having a grooved surface (3-6 μm space, 15-20 μm width and 5 μm depth) (the template) in an environmental chamber under conditions of controlled humidity and temperature then maintained at those conditions during evaporation. Under conditions tested, this typically occurred within 20-30 mins.

The pattern template was manufactured as described in WO2012/038691 A1 [3], which is herein incorporated by reference in its entirety.

The films were cast on the silicon substrate under the following different conditions:
(1) (Type I)—low temperature, low relative humidity
(2) (Type II)—higher relative humidity
(3) (Type III)—higher temperature, significantly higher humidity

The polymer films were peeled away from the template, washed in distilled H₂O and each side sterilized by UV irradiation for 30 minutes prior to testing.

Complete solvent evaporation was confirmed by FTIR (Thermo Nicolet Nexus™ FTIR (Cambridge, UK) controlled by OMNIC Software Version 6.1a), which ensured that no solvent toxic effect would occur in the subsequent cell growth and testing.

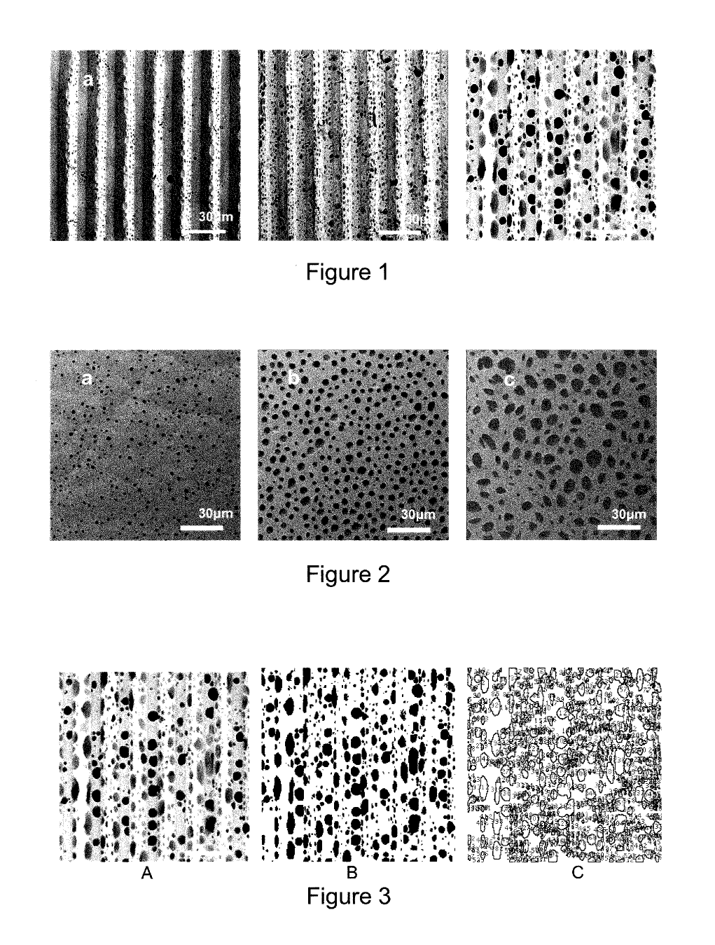

These results are shown by SEM images FIGS. 1 and 2. FIG. 1 shows the grooved and pitted “surface side” of the cast film. FIG. 2 shows the pitting on the ungrooved “air side” of the cast film.

### Surface Analysis—SEM

The surface topography of the solvent cast films (three samples per pattern) was characterized by scanning electron microscopy (SEM). After mounting film samples on stubs, vacuum sputtering gold coater (Edward, UK) was used to coat film samples. The coated films were then imaged by a desktop SEM (Phenom World G2, The Netherlands).

As shown in FIG. 1, the grooved pattern was successfully transferred on to the surface of PCL/PLA films during casting. During the production of the films, DCM gradually evaporated from the cast films with different evaporation rates, the evaporation rate being determined by the humidity and temperature conditions used in the environmental chamber, leading to the formation of the films with different sizes of pits and pitted area coverage. When fixing the temperature and increasing the humidity the rate of DCM evaporation became lower, resulting in films having larger pits and greater pitted area coverage. Further increasing the temperature and humidity, larger pits and greater pitted area coverage of casting film were observed. The experimental results show that environmental temperature and humidity affected the surface structure and morphology of solvent casting films due to changing the solvent evaporation rate. Three sets of grooved films were prepared under the various conditions, and the average pits sizes of these resultant films were measured from SEM images by Image J software. These measurements are provided in Table 1:

TABLE 1
Average pit size and Percentage Pitted Area Coverage for films
cast on grooved silicon substrate (width 15-20 μm; depth
5 μm; space 3-6 μm).

| | Average Pit Size | Percentage Pitted |
|---|---|---|
| Film | (μm)² | Area (%) |
| Type I | 0.5 | 8 |
| Type II | 1.2 | 23 |
| Type III | 4.3 | 42 |

For films prepared under Type III conditions (high temperature and humidity), there were several macro-sized pores, i.e. through holes, (not shown in the figures, sized up to 0.75 (mm)²) which were visible to the naked eye. Throughout, the measurements of average pit size and percentage of pitted area given focus on pits (not pores) under 1000 (μm)² in area. Any pores and pits over this area are discounted.

To measure the average pit size and % pitted area, three films made under each of the Type I, Type II, and Type III conditions were prepared for SEM imaging (mounting onto the SEM stud and coated with gold). For each film Type, three random surface areas were chosen for SEM imaging. The SEM images were processed firstly to give an 8-bit image (FIG. 3a), then a threshold was applied to detect only the pits (FIG. 3b). Finally, automatic measurements were obtained (FIG. 3c).

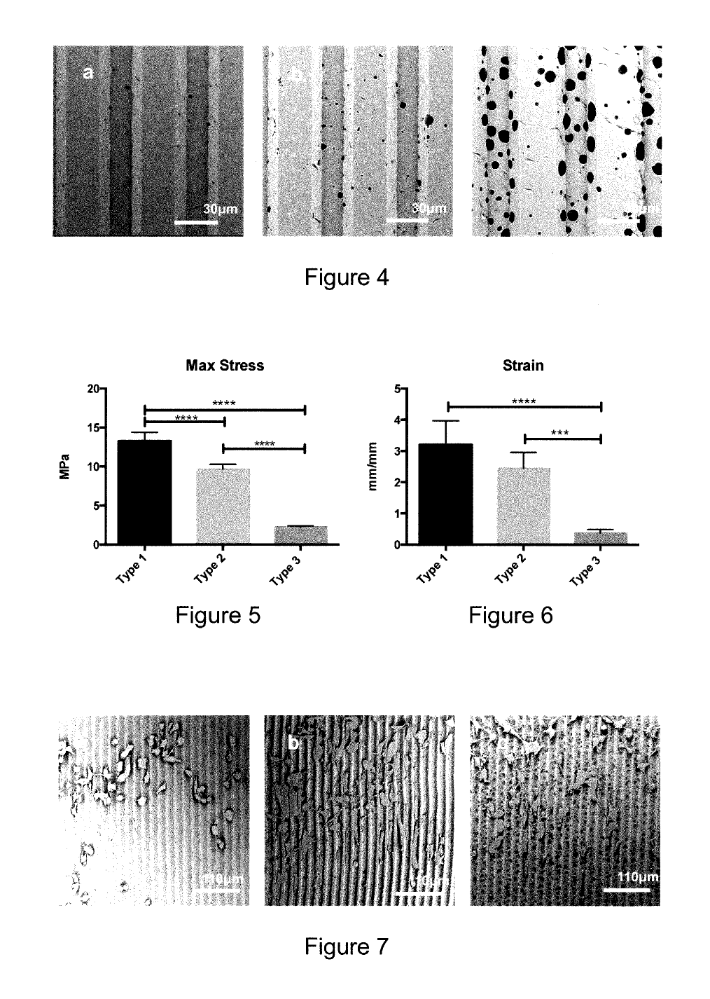

The present inventors have found that the not only humidity and temperature but also the geometrical pattern of the template affects the pit size. Another truncated V-shaped grooved silicon substrate (silicon substrate with width 25-30 μm, depth 10 μm and space 20-25 μm) was employed to compare the results with the optimized solvent-cast grooved silicon substrate (width 15-20 μm; depth 5 μm; space 3-6 μm) (FIG. 4). As shown by FIGS. 1 and 4, the casting films made on these two templates exhibited different pits and porosity under the same temperature and humidity preparation conditions. The average of pit size and % pitted area of the films cast on this silicon substrate are shown in Table 2.

TABLE 2
Average pit size and Percentage Pitted Area Coverage for films
cast on grooved silicon substrate (width 25-30 μm; depth
10 μm; space 20-25 μm).

| | Average Pit Size | Percentage Pitted |
|---|---|---|
| Film | (μm)² | Area (%) |
| Type I* | 0.26 | 0.11 |
| Type II* | 0.46 | 1.28 |
| Type III* | 6.2 | 9 |

The present inventors attribute this observation to the influence of geometric pattern on the evaporation rate of the solvent, thereby generating different average pit sizes and % pitted area coverage. The experimental results indicated that the geometry of the silicon substrates used to cast the films provides another route to control the surface structure and morphology of the solvent cast films. Elsewhere throughout the application, the labels Type I, Type II, and Type III (without the asterisk) refer to films cast on grooved silicon substrate (width 15-20 μm; depth 5 μm; space 3-6 μm).

### Mechanical Testing

The tensile strength and maximum strain of the films were measured, and the strength of the films compared to native peripheral nerve.

Tensile strength is defined as the maximum amount of tensile stress that a material can be subjected to before failure. Maximum strain is measured as the total elongation per unit length of material subject to same applied stress.

Tensile strength and maximum strain were measured on a mechanical tensile tester (lnstron 1122) at 23±0.1° C., 50%±2% relative humidity. The film samples were cut into dumbbell shapes with a gauge length of 10 mm, a width of 2 mm, and a thickness of around 50-80 μm. Before testing, the samples were left in a climate-controlled laboratory for 24 h at a temperature of 23.0±0.1° C. and a relative humidity of 50±5%. The specimens were deformed at a loading rate of 10 mm/min with a 0.010 KN scale load. Five samples of each pattern were analyzed and the experiment was repeated in triplicate.

The tensile strength was the average of ultimate stress at the breaking point of the films.

The results are set out in Table 3 and FIGS. 5 and 6:

TABLE 3
Mechanical strength of the Type I, Type II, and Type III films.

| Film | Strength (MPa) | Strain (mm/mm) |
|---|---|---|
| Type I | 13.28 ± 1.1 | 3.20 ± 0.76 |
| Type II | 9.62 ± 0.65 | 2.44 ± 0.51 |
| Type III | 2.2 ± 0.17 | 0.37 ± 0.11 |

It can be seen that the temperature and humidity conditions used to form the film, in addition to affecting the size of distribution of the pits, have significant effects on the mechanical properties of the films. PCL/PLA films prepared under low temperature and humidity conditions achieved the highest maximum stress and strain, and Young's modulus. Preparing the films at the same temperature but at higher humidity led to a relative decrease in max stress and strain, and Young's modulus. Films prepared under conditions of higher temperature and humidity exhibited even lower maximum stress and strain, and Young's modulus.

It has been reported that the max stress of acellular nerve is 1.4 MPa in comparison to 2.72 MPa for fresh rat nerve, respectively [4]. Of the films tested, the films prepared under lower temperature and humidity conditions (Type I and Type II) have been shown to exceed significantly the acellular nerve and fresh nerve in these tests. The films casted under the higher temperature and humidity conditions (Type III) were weakest, outweighing acellular nerve and being slightly below fresh nerve in these tests.

### Cell Culture and Cell Proliferation

NG108-15 cells (neuroblastoma×rat glioma hybrid cells, European Collection of Cell Culture) were cultured in DMEM with 10% fetal bovine serum at 37° C. and 5% CO₂. Prior to in vitro experiments, each side of each film sample (three samples of each pattern, all cast on patterned silicon (width 15-20 μm; depth 5 μm; space 3-6 μm)), which were fixed on the scaffdex (Scaffdex Cellcrown, Finland) to prevent floating from well bottom, was sterilized under UV light for 30 minutes in a class II microbiological safety cabinet (Envair, UK).

### SEM Fixation

One-milliliter NG108-15 cells (3,000/well) were seeded on the sterilized films (three samples of each pattern) in 24 well plates and cultured for 24 hours, 72 hours and 120 hours. The cell seeded samples were rinsed with PBS twice and then fixed with 1.5% gluteraldehyde at 4° C. Then, the cell seeded samples were dehydrated by the gradient of ethanol solution (50%, 70%, 90% and 100%) and HMDS. Finally, the images were acquired using desktop SEM after gold coating as described herein.

Alamar blue assay was used to analyze the proliferation rate of NG 108-15 cells by redox reaction. One-milliliter NG108-15 cells (5,000/well) were seeded on the sterilized films (each pattern in triplicate) in 24 well plate. 100 μl Alamar blue solution was added to each well at timepoints at 24 hours, 72 hours and 120 hours. After 2.5 hours of incubation at 37° C. and 5% CO₂, 200 μl of media was transferred to a 96-well plate in triplicate, then fluorescence was measured at 530-510 nm excitation and 590 nm emission.

Fluorescent staining was used to identify and stain cytoskeleton structure. F-actin can be stained in green by phalloidin and nucleus can be stained in blue by DAPI. One-milliliter NG108-15 cells (3,000/well) were seeded on the sterilized films (each pattern in triplicate) in 24 well plates and cultured for 24 hours, 72 hours and 120 hours in incubation at 37° C. and 5% CO₂. The cell seeded samples were washed by PBS before fixing with parafomaldehyde (PFA) for 15 minutes. After 0.1% Triton was added for 10 minutes at room temperature, PBS-bovine serum albumin solution (1 mg/ml BSA) was used for 1 hour incubation. PBS-phalloidin solution (40:1) was added to each sample and the samples incubated for 20 minutes at room temperature in the dark. Finally, the samples were washed by PBS three times and mounted on glass slides with ProLong Gold antifade reagent with DAPI. Images were taken with Leica TCS SP5 confocal at 405 nm excitation and 488 nm emission wavelength.

As shown in FIG. 7, the cells were attached on day 1 and then proliferated significantly throughout 5 days post seeding. For the first day of cell attachment, the Type III films proved least amenable to cell attachment compared to the Type I and Type II films. Without wishing to be bound by any particular theory, the present inventors believe that pitted area and larger pits of these films do not provide sufficient contact area for cell attachment.

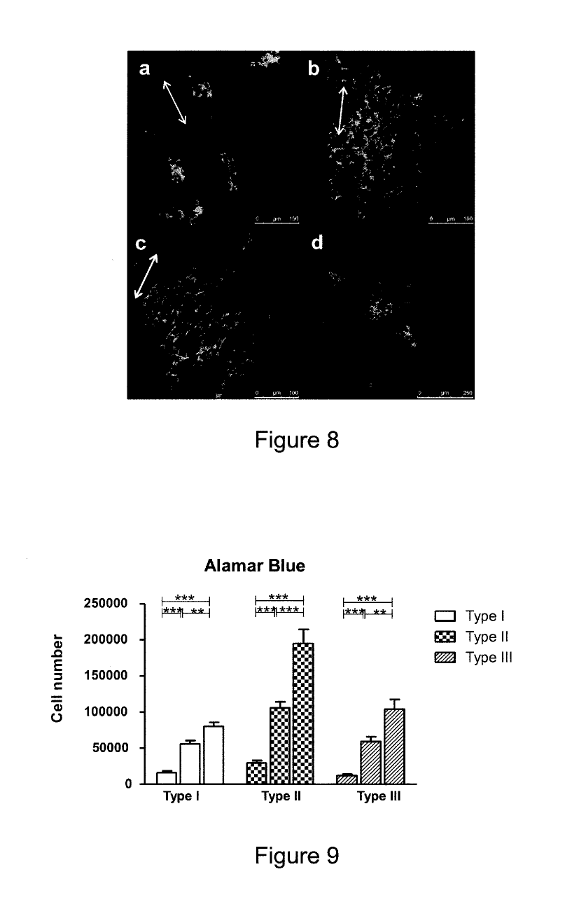

FIG. 8 shows the fluorescence micrograph of NG108-15 on the Type I, Type II and Type III films, and a smooth glass slide control, on day 5 following an initial cell seeding density of 3000 cell/ml.

The results showed the Type II films showed the best initial cell adhesion, the number of cells on day one being over 2.5 times that of Type III and almost twice that of Type I. The Type I films having an average pit size of 0.5 (μm)² and 8% pitted area demonstrated the lowest number and growth rate of cells on day 3 (241.94%), with the Type II films significantly outperforming both the Type I and III.

The Type II films also achieved the largest number of cell on day 5, followed by the Type III and then the Type I films. In all the repeated three experiments, the film with 1.2 (μm)² average pit size and 23% pitted area (Type II) showed better cell growth on the day 5 than other two films. The results are shown in FIG. 9.

The above results show that the combination of microgrooves and pits tailored to the microgrooved structure results in scaffolds having impressive performance in terms of cell attachment, growth, and proliferation, and desirable mechanical properties for use. This balance of properties to satisfy a number of challenging criteria results in improved scaffolds for peripheral nerve growth and repair.

### REFERENCES

A number of publications are cited herein in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
[1] Kalbermatten, D. F. et al., “Fibrin matrix for suspension regenerative cells in an artificial nerve conduit”, Journal of Plastic, Reconstructive & Aesthetic Surgery (2008), Volume 61, Issue 6, Pages 669-675.
[2] WO2010/029295 A2.
[3] WO2012/038691 A1.
[4] Borschel, G. H., et al, Mechanical properties of acellular peripheral nerve. Journal of Surgical Research (2003), Volume 144, Issue 2, Pages 133-139.

1. A peripheral nerve growth scaffold including poly-ε-caprolactone (PCL) wherein a surface of the scaffold comprises pits (blind holes) and microgrooves, the pits covering at least 5% of the surface.

2. A peripheral nerve growth scaffold including poly-ε-caprolactone (PCL) wherein a surface of the scaffold comprises pits (blind holes) and microgrooves, the pits having an average area equal to or greater than 0.5 (μm)².

3. The peripheral nerve growth scaffold according to claim 1, wherein the pits cover at least 5% of the surface.

4. (canceled)

5. The peripheral nerve growth scaffold according to claim 1, wherein the pits cover at least 20% of the surface.

6. The peripheral nerve growth scaffold according to claim 1, wherein the % coverage of the pits on the grooved surface is in the range 18-30%.

7. The peripheral nerve growth scaffold according to claim 1, wherein the % coverage of the pits on the grooved surface is in the range 20-26%.

8. The peripheral nerve growth scaffold according to claim 1, wherein the pits have an average area equal to or greater than 0.9 (μm)².

9. The peripheral nerve growth scaffold according to claim 1, wherein the pits have an average area in the range 0.9-3.0 (μm)².

10. (canceled)

11. The peripheral nerve growth scaffold according claim 1, wherein the thickness of the scaffold is in the range 50-80 μm.

12. The peripheral nerve growth scaffold according claim 1, wherein the microgrooves have a cross-sectional profile having sloping walls.

13. The peripheral nerve growth scaffold according to claim 1, wherein the microgrooves have an average width in the range 15-20 μm.

14. The peripheral nerve growth scaffold according claim 1, wherein the microgrooves have an average depth in the range 3-7 μm.

15. The peripheral nerve growth scaffold according to claim 1, wherein the microgrooves have an average spacing in the range 3-6 μm.

16. The peripheral nerve growth scaffold according to claim 1, wherein the surface has microgrooves having an average width in the range 15-20 μm, an average depth of about 5 μm, and an average spacing in the range 3-6 μm, and pits having an average in the range 0.9-3.0 (μm)² with 18-30% coverage.

17. (canceled)

18. The peripheral nerve growth scaffold according to claim 1, wherein the nerve growth scaffold also includes polylactic acid (PLA), the PCL and PLA being present in a blend.

19. The peripheral nerve growth scaffold according to claim 1, wherein the scaffold is a tubular conduit.

20. The peripheral nerve growth scaffold according to claim 19, wherein the tubular conduit wall has a thickness in the range 10 μm to 300 μm.

21. A method of treating a damaged peripheral nerve comprising attaching the peripheral nerve scaffold of claim 1 to the damaged peripheral nerve.

22. A method of making a peripheral nerve growth scaffold according to claim 1, the method comprising the steps of:
i) selecting a template forming a negative of a microgroove pattern;
ii) applying a solution comprising poly-ε-caprolactone (PCL) to the template;
iii) evaporating the solvent to form a film under controlled temperature and humidity conditions in an environmental chamber to control the formation of pits at the surface; and
iv) removing the film from the template.

23. The method of claim 22, wherein the method further comprises the step of:
v) joining opposite edges of the film together by heat sealing to form a tubular conduit.
